(19) United States
(12) Patent Application Publication
(10) Pub. No.: US 2012/0302738 A1
(43) Pub. Date: Nov. 29, 2012

(54) Recombinant antibody vector

(75) Inventors: Trent Phillip Munro, Chapel Hill (AU); Martina Louise Jones, Narangba (AU); Matthew George Smede, Taringa (AU)

(73) Assignee: ACYTE BIOTECH PTY LTD, St. Lucia QLD (AU)

(21) Appl. No.: 13/510,269

(22) PCT Filed: Nov. 16, 2010
(86) PCT No.: PCT/AU10/01532

§ 371 Date: Aug. 3, 2012

(30) Foreign Application Priority Data
Nov. 16, 2009 (AU) 2009905601

(51) Int. Cl. C12N 15/63; C12P 19/34; C12N 9/12; C07K 16/00; C12N 1/21; C12N 5/10; C12N 1/19; C12P 21/00; C12N 9/18; C12N 15/66

(52) U.S. Cl. 530/387.3; 435/320.1; 435/197; 435/91.2; 435/194; 435/91.41; 435/252.3; 435/419; 435/254.2; 435/326; 435/69.6

(57) ABSTRACT

A recombinant antibody vector for producing a single chain recombinant antibody comprises: (a) a contiguous nucleotide sequence: (i) that comprises a restriction endonuclease site that encodes an amino acid sequence of an immunoglobulin variable region; and (ii) that encodes an immunoglobulin constant region amino acid sequence in the same reading frame as (i), wherein another nucleotide sequence encoding (iii) an immunoglobulin variable region amino acid sequence, is insertable into the restriction endonuclease site in the same reading frame as (ii); and (b) one or more regulatory nucleotide sequences operably linked or connected to said nucleotide sequence, wherein the amino acid sequence in (i) comprises amino acids conserved in different immunoglobulin variable regions. The restriction endonuclease site may be a SacI site which encodes the conserved amino acids glutamate and leucine. In frame insertion of the nucleotide sequence of (iii) is facilitated by ligase independent cloning.

### CROSS-REFERENCE TO RELATED APPLICATIONS

This patent application is a the U.S. national stage of International Patent Application No. PCT/AU2010/001532, filed Nov. 16, 2010, designating the United States, and claiming priority to Australian Patent Application No. 2009905601, filed Nov. 16, 2009, which are incorporated by reference.

### SEQUENCE LISTING

Incorporated by reference in its entirety herein is a computer-readable nucleotide/amino acid sequence listing submitted concurrently herewith and identified as follows: 16,901 byte ASCII (text) file named “710467SequenceListing.txt” created on Aug. 3, 2012.

### FIELD

THIS INVENTION relates to nucleic acid vectors for producing recombinant antibodies, particularly single chain recombinant antibodies.

### BACKGROUND

Over the past 10-15 years there has been a surge of interest in the use of recombinant monoclonal antibodies (mAbs) as therapeutic agents. In 2007, mAb sales in the USA alone exceeded $14 billion, with a year on year growth rate of 22% (Aggarwal, 2008). With the number of approved mAbs approaching 30 and hundreds of new candidates in the pipeline, this trend shows no signs of slowing. Most therapeutic recombinant mAbs are members of the IgG family and owing to their large size and complex glycosylation patterns, these molecules are currently produced in mammalian cells, with the vast majority utilizing Chinese Hamster Ovary (CHO) cells as the production host (Wurm, 2004).

The path from discovery to the clinic for a therapeutic, recombinant mAb can be a long and tedious process, often taking several years. The first step of this process involves identification of a high-affinity binder to a target molecule, such as a surface antigen over-expressed during tumourigenesis. Considerable effort has been dedicated to elucidating methods that facilitate isolation of binding moieties to an antigen of interest. The first mAbs were produced utilizing hybridoma technology, however the resultant murine antibodies are not suitable for therapeutic applications (Berger et al., 2002). Subsequently, methods such as CDR grafting, phage, yeast and ribosome display were developed (for review see: (Hoogenboom, 2005)). Phage display is the most commonly used method. This technique identifies single chain variable fragment (scFv) or fragment antigen binding (Fab) elements, that bind to the target molecule isolated from libraries of high-complexity, emulating the naïve immune repertoire. This library may contain murine or human sequences and more recently, completely synthetic libraries have been created. Crucially, since these fragments contain antibody variable regions, they require “reformatting” into an expression vector containing both the requisite constant region sequences and the elements for high-level expression in mammalian cells. This reformatting step can be a protracted and complicated process since the sequences of the isolated fragments are by nature variable. This makes traditional PCR and/or restriction endonuclease cloning problematic. For example an anti-TNF antibody isolated from a naïve Fab immunoglobulin gene library was rebuilt as a complete antibody by a tripartite ligation; a fragment containing the leader sequence and the amino terminus of the V (variable) domain, a second fragment containing the remainder of the V domain and Cλ constant region, and the expression vector. The reformatting required PCR using fragment specific primers and appendage of compatible restriction sites (Mahler et al., 1997). Existing antibody reformatting vectors exhibit limited flexibility and the codons formed by restriction endonuclease recognition sequences often result in the addition of several “foreign” amino acids into the primary sequence (Coloma et al., 1992; Persic et al., 1997; Jostock et al., 2004).

### SUMMARY

The invention relates to a vector for recombinant antibody production which eliminates, or at least appreciably minimizes, the presence of “foreign” or “extraneous” amino acids in an expressed recombinant antibody that can compromise antigen binding by recombinant antibody.

In a broad form, the invention provides a recombinant antibody vector for a single chain recombinant antibody, the vector comprising a nucleotide sequence that encodes an amino acid sequence that is at least partly conserved in a plurality of different immunoglobulin variable regions and which is encoded by a restriction endonuclease site into which can be inserted a nucleotide sequence encoding an immunoglobulin variable region.

In a first aspect, the invention provides a recombinant antibody vector comprising a nucleotide sequence that encodes: (i) an amino acid sequence of an immunoglobulin variable region which is encoded by a restriction endonuclease site; and (ii) an immunoglobulin constant region amino acid sequence; wherein the nucleotide sequence further comprises one or more regulatory nucleotide sequences operably linked or connected to said nucleotide sequence.

Suitably, another nucleotide sequence encoding (iii) an immunoglobulin variable region amino acid sequence is insertable into the recombinant antibody vector in the same reading frame as (ii), preferably without encoding one or more amino acids other than those in (i), (ii) and (iii).

In a preferred embodiment, the amino acid sequence in (i) comprises a plurality of amino acids that are at least partly conserved in different immunoglobulin variable regions.

Typically, the amino acid sequence in (i) comprises, or consists of, two amino acids.

Preferably, a first amino acid of the amino acid sequence in (i) is glutamate (E). Preferably, a second amino acid of the amino acid sequence in (i) is leucine (L). More preferably, the amino acid sequence in (i) consists of EL.

Preferably, according to the first aspect, the restriction endonuclease site is a SacI site.

Suitably, the immunoglobulin constant region amino acid sequence of (ii) and the immunoglobulin variable region amino acid sequence of (iii) are of, or from, different immunoglobulin molecules.

Suitably, said nucleotide sequence of the recombinant antibody vector further encodes (iv) a signal peptide amino acid sequence.

It will be appreciated that the amino acid sequences in (ii), (iii) and (v) may be fragments of immunoglobulin constant regions, variable regions and signal peptides, respectively.

This aspect of the invention also provides a recombinant antibody expression construct comprising the recombinant antibody vector and said nucleotide sequence in (iii) encoding the immunoglobulin variable region amino acid sequence.

In a second aspect, the invention provides a kit comprising the recombinant antibody vector of the first aspect and one or more reagents for insertion of another nucleotide sequence encoding an immunoglobulin variable region amino acid sequence into the vector.

The one or more reagents may include a restriction endonuclease.

Preferably, the restriction endonuclease site is SacI.

The one or more reagents may include an enzyme and optionally one or more other reagents, for ligase independent cloning (LIC) of the nucleotide sequence into the vector.

In a third aspect, the invention provides a method of producing a recombinant antibody expression construct including the step of inserting another nucleotide sequence that encodes an immunoglobulin variable region amino acid sequence into the recombinant antibody expression vector of the first aspect.

Preferably, the nucleotide sequence that encodes an immunoglobulin variable region amino acid sequence is inserted by ligase independent cloning (LIC).

In a fourth aspect, the invention provides a recombinant antibody expression construct produced according to the method of the third aspect.

In a fifth aspect, the invention provides a host cell comprising the recombinant antibody vector of the first aspect or the recombinant antibody expression construct of the fourth aspect.

In a sixth aspect, the invention provides a method of producing a recombinant antibody including the step of isolating, purifying or enriching a recombinant antibody from the host cell of the fourth aspect.

In a seventh aspect, the invention provides a recombinant antibody encoded by the recombinant antibody expression construct of the first aspect or the fourth aspect.

Throughout this specification, unless the context requires otherwise, the words “comprise”, “comprises” and “comprising” will be understood to imply the inclusion of a stated integer or group of integers but not the exclusion of any other integer or group of integers.

### BRIEF DESCRIPTION OF THE FIGURES

FIG. 1. mAbXpress vector system. Vectors contain all required elements for high-level expression in mammalian cells as well as the backbone sequence of the IgG including a secretory signal peptide. The E-L codons form a SacI site for vector linearization prior to In Fusion™ mediated cloning of the Variable region. The Variable region PCR product contains 15 by at the 3′ and 5′ with exact homology to the destination vector insertion site flanking the SacI site.

FIG. 2. Analysis of the expression and purification of 3C12 IgG1.

(A) Non-reduced (NR) and 2-mercaptoethanol reduced (R) samples of culture supernatants (i, iii) and 5 μg affinity purified material (ii, iv) were separated on 4-12% SDS-PAGE and stained with Coomassie Blue R250. (B) Analytical size exclusion chromatography of protein-A purified recombinant 3C12 antibody and gel filtration standards. The sample shows no detectable aggregation and an as predicted molecular weight of 145 kDa.

FIG. 3. Functional analysis of purified 3C12, an anti-human CD83 IgG. (A) 25 μg/ml 3C12 IgG1 binds CD83+ cell lines KM-H2, L428 and FDCP1 cells transfected with human CD83. No difference between 3C12 and an isotype IgG1 control is seen on un-transfected FDCP1 cells (CD83−). (B) 3C12 IgG1 induced significant lysis of the KM-H2 cell line relative to Herceptin (negative control) via a CD16-dependent mechanism in vitro. Error bars represent standard error of the mean.

FIG. 4. Plasmid maps showing features required for expression and selection in mammalian cells. A) Example of IgG heavy chain and B) light chain. In each case the variable region insertion point is indicated.

### DETAILED DESCRIPTION

The present invention has arisen from the inventors' realization of a need for a recombinant antibody expression system that minimizes the complexity of amplification of nucleotide sequences encoding immunoglobulin variable regions, preferably provides ligase independent cloning of amplified nucleotide sequences into the vector and eliminates or minimizes the inclusion of foreign or extraneous amino acids that can lead to reduced antigen binding. Assisting the development of a recombinant antibody vector that addresses this need was the inventors' discovery that the amino acid glutamate (E) occurs at or near the N-terminus of about 10% of immunoglobulin variable regions and that the amino acid leucine (L) occurs at or near the C-terminus of about 10% of immunoglobulin variable regions. The inventors have created a vector comprising the nucleotide sequence GAGCTC (SEQ ID NO:1) encoding the amino acid sequence EL, which provides a recognition and cleavage site for the restriction endonuclease SacI. By including this nucleotide sequence, the recombinant antibody vector provides a convenient linearization site for insertion of a nucleotide sequence encoding an immunoglobulin variable region so that the E residue is N-terminal of the immunoglobulin variable region and the L residue is C-terminal of the immunoglobulin variable region. This positioning of the partly conserved E and L residues is present in a significant proportion of immunoglobulin variable regions and thereby would be less likely to negatively affect antigen recognition and binding.

Accordingly, in one preferred aspect, the invention provides a single chain recombinant antibody vector comprising: (a) a nucleotide sequence: (i) that comprises a restriction endonuclease site that encodes an amino acid sequence of an immunoglobulin variable region; and (ii) that encodes an immunoglobulin constant region amino acid sequence in the same reading frame as (i), wherein another nucleotide sequence encoding (iii) an immunoglobulin variable region amino acid sequence, is insertable into the restriction endonuclease site in the same reading frame as (ii); and (b) one or more regulatory nucleotide sequences operably linked or connected to said nucleotide sequence.

Suitably, said another nucleotide sequence encoding (iii) an immunoglobulin variable region amino acid sequence is insertable into the recombinant antibody vector in the same reading frame as (ii), preferably without encoding one or more amino acids other than those in (i), (ii) and (iii).

The invention also provides a recombinant antibody expression construct comprising the recombinant antibody vector and said nucleotide sequence in (iii) encoding the immunoglobulin variable region amino acid sequence.

Suitably, the immunoglobulin constant region amino acid sequence of (ii) and the immunoglobulin variable region amino acid sequence of (iii) are of, originate or derived from, different, separate or distinct (i.e. not the same) immunoglobulin molecules.

Accordingly, the recombinant antibody vector provides a “generic”, “platform” or “backbone” immunoglobulin constant region into which can be included or grafted an immunoglobulin variable region of interest.

As used herein, a “vector” is an artificially created nucleic acid molecule that suitable for manipulation, propagation and/or expression of a nucleotide sequence of interest. Vectors may be plasmids, artificial chromosomes, phagemids, cosmids or genetically-modified viruses, although without limitation thereto. An “expression construct” is a vector into which has been inserted a nucleotide sequence to be expressed.

The term “nucleic acid” includes DNA and RNA, inclusive of single and double-stranded forms.

Preferably, the vector is a double stranded DNA plasmid.

Typically, the restriction endonuclease recognition site comprises six (6) contiguous nucleotides that encode two amino acids of an immunoglobulin variable region.

The vector preferably comprises a nucleotide sequence of a restriction endonuclease recognition site that encodes amino acids that are at least partly conserved in a plurality of different immunoglobulin variable regions. Preferably, the amino acids are present in at least 1%, 2%, 3%, 4%, 5%, 6%, 7%, 8%, 9% or 10% of immunoglobulin variable regions. In a preferred embodiment, the respective amino acids are conserved at or near the N- or C-termini of the different immunoglobulin variable regions.

In a particularly preferred embodiment, the amino acid sequence is EL and is encoded by the nucleotide sequence GAGCTC (SEQ ID NO:1), which provides a recognition and cleavage site for the restriction endonuclease SacI. The N-terminal E amino acid and the C-terminal L amino acid are present in about 10% of immunoglobulin variable region amino acid sequences.

However, it will be appreciated that other nucleotide sequences, preferably comprising or consisting of a nucleotide sequence that encodes two or three amino acids, and that forms a restriction endonuclease recognition site may be particular for an antibody variable region amino acid sequence, without necessarily being conserved or present in other antibody variable region amino acid sequences.

By the term “protein” is meant an amino acid polymer, which may comprise natural or non-natural amino acids, D- or L-amino acids. Generally, a “peptide” is a protein having no more than 60 contiguous amino acids.

As used herein an “antibody” is an immunoglobulin protein capable of specifically binding an antigen and at least comprises an amino acid sequence of an immunoglobulin constant region and an amino acid sequence of an immunoglobulin variable region. These amino acid sequences may constitute all or a portion or fragment of the entire amino acid sequence of the respective immunoglobulin constant region and immunoglobulin variable region from which they were originally derived. Suitably, the immunoglobulin variable region fragment is capable of binding an antigen or epitope.

The term “immunoglobulin constant region” includes within its scope immunoglobulin heavy chain and light chain constant regions and fragments thereof of mouse or human origin. A fragment may constitute at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90% or 91%, 92%, 93%, 94%, 95%, 96%, 97%, 98% or 99% of an entire immunoglobulin constant region.

Non-limiting examples of constant region sequences are provided in the Examples, although other examples of constant regions may be found by searching sequence databases at NCBI or dedicated databases such as Kabat or V BASE.

The term “immunoglobulin variable region” includes within its scope immunoglobulin heavy and light chain variable regions of mouse or human origin.

Heavy chains may be of any isotype including IgM, IgG, IgD, IgE and IgA or any subtype including IgG₁, IgG₂, IgG₂ₐ, IgG₃ and IgG₄.

Light chains may be λ or κ light chains.

The immunoglobulin variable region or fragment thereof suitably includes sufficient amino acid sequence to specifically bind an antigen or an epitope. Typically, the immunoglobulin variable region includes at least one “complementarity-determining region (CDR)”, or fragment thereof, which refers to the hypervariable regions in each of the heavy and light chains that are primarily responsible for binding to an epitope of an antigen. In this context, a fragment may constitute at least 10%, at least 15%, at least 20%, at least 25%, at least 30%, at least 35%, at least 40%, at least 50%, at least 55%, at least 60%, at least 65%, at least 70%, at least 75%, at least 80%, at least 85%, at least 90% or 91%, 92%, 93%, 94%, 95%, 96%, 97%, 98% or 99% of an entire CDR or entire variable region.

The CDRs of each chain are typically referred to as CDR1, CDR2, and CDR3, numbered sequentially starting from the N-terminus.

Preferably, the immunoglobulin variable region includes comprises three CDRs of the same immunoglobulin variable region.

Suitably, the recombinant antibody vector further comprises a nucleotide sequence encoding a signal peptide amino acid sequence. Non-limiting examples of signal peptide sequences are provided in the Examples, although other examples of signal peptide sequences may be found at the Signal Peptide Website.

In a particularly preferred embodiment, another nucleotide sequence encoding an immunoglobulin variable region amino acid sequence is insertable into the recombinant antibody vector in the same reading frame as the nucleotide sequence encoding the immunoglobulin constant region amino acid sequence and the signal peptide amino acid sequence, without resultantly encoding one or more additional amino acids other than those encoded by the restriction endonuclease site and those encoded by the inserted nucleotide sequence encoding the immunoglobulin variable region amino acid sequence and encoded by the nucleotide sequence encoding the signal peptide and the immunoglobulin constant region amino acid sequence.

Accordingly, in a preferred embodiment of the recombinant antibody expression construct, said nucleotide sequence in (a) encodes a contiguous amino acid sequence comprising or consisting of, sequentially: a first amino acid of the amino acid sequence in (i) the amino acid sequence of (iii), a second amino acid of the amino acid sequence in (i), and the amino acid sequence of (ii). Preferably, the first amino acid is E and the second amino acid is L.

The recombinant antibody vector and expression construct suitably comprises one or more regulatory nucleotide sequences.

By “regulatory nucleotide sequences” is meant nucleotide sequences that facilitate initiation, control or termination of transcription, post-transcriptional processing, splicing, translation or other events associated with expression of said nucleotide sequence.

Non-limiting examples of regulatory nucleotide sequences include promoters, polyadenylation sequences, enhancers, introns, ribosomal binding sites, splice donor/acceptor sites, translation start and/or termination sequences and the like.

The choice of regulatory nucleotide sequences will be somewhat dependent upon the origin of the host cell or organism in which a recombinant antibody is to be expressed. Such regulatory nucleotide sequences are well known in the art.

The recombinant antibody vector suitably comprises a promoter operably linked or connected to the nucleotide sequence encoding the antibody. The promoter may be constitutive, regulatable (i.e. inducible or repressible), tissue specific or subject to other desired functional constraints or influences on promoter activity. In embodiments relating to mammalian cell expression, the promoter may be any promoter useful in mammalian expression systems, including but not limited to a CMV promoter, an SV40 promoter, an elongation factor α promoter (e.g. pEF-BOS), a crystallin promoter (e.g. αA crystallin, β2 crystallin) or a hybrid promoter (e.g. SRα), for example.

In a preferred embodiment, the recombinant antibody vector comprises a CMV promoter.

The recombinant antibody vector may also include one or more selectable marker genes to allow the selection of transformed host cells in media comprising a selection agent. Generally, selectable marker genes confer resistance to selection agents such as ampicillin, kanamycin, tetracycline, chloramphenicol, neomycin, geneticin, streptomycin and gentamycin, although without limitation thereto. Suitable genes are readily available in the art. Generally, a selectable marker gene is included to facilitate selection of transformed bacteria for bacterial propagation of the recombinant antibody vector. However, in some embodiments, another selectable marker gene may be included to facilitate selection of transformed host cells used for expression of the recombinant antibody. Typically, the selectable marker gene will be operably linked to a promoter suitable for expression of the selectable marker gene in a desired host cell.

It will also be appreciated that to facilitate recombinant manipulation and propagation in bacterial host cells, the recombinant antibody vector may comprise a bacterial origin of replication, such as an f1 bacteriophage, colE1 or pUC origin of replication.

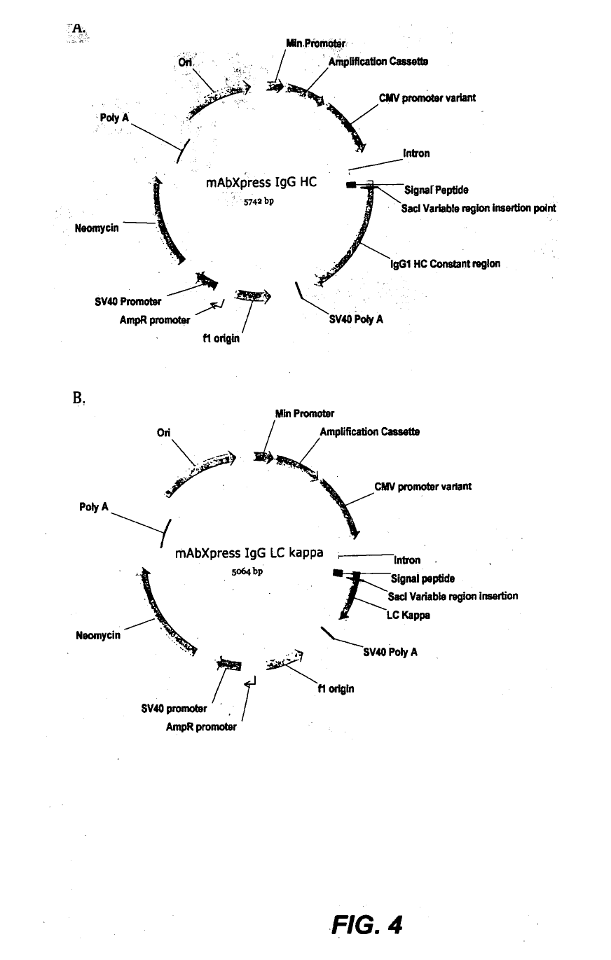

Non-limiting examples of particular recombinant antibody plasmid vectors are shown in FIG. 4A and FIG. 4B.

The recombinant antibody vector of the invention allows for the production of recombinant antibodies comprising virtually any variable region amino acid sequence with a relatively simple “one-step” amplification and cloning system that does not introduce extraneous amino acids that potentially affect antigen recognition and binding. Variable region amino acid sequences may be sourced from phage display libraries of scFv fragments or Fab fragments, ribosome and mRNA display libraries, microbial cell display libraries or libraries produced by directed evolution (such as reviewed in Hoogenboom, 2005), although without limitation thereto.

Alternatively, variable region amino acid sequences may be sourced from an antibody-producing hybridoma or other cell expressing a nucleic acid molecule encoding an immunoglobulin variable region amino acid sequence.

Advantageously, the recombinant antibody vector of the invention facilitates insertion of a nucleotide sequence encoding a variable region immediately 5′ of the nucleotide sequence encoding the constant region without addition of any extraneous amino acid-encoding nucleotide sequence.

In a further aspect, the invention provides a method of producing a recombinant antibody expression construct including the step of inserting another nucleotide sequence that encodes an immunoglobulin variable region amino acid sequence into the recombinant antibody expression vector as hereinbefore described.

Typically, a nucleotide sequence encoding said immunoglobulin variable region would be amplified from a library or other source by a nucleotide sequence amplification technique such as PCR.

Generally, a single pair of forward and reverse PCR primers would comprise:
Forward 5′ to 3′: at least 10, 11, 12, 13, 14, 15, 16, 17, 18, 19, 20 or more contiguous nucleotides of a nucleotide sequence immediately 5′ of the restriction endonuclease site in the recombinant antibody vector; and a 5′ portion of the nucleotide sequence encoding a variable region.
Reverse 5′ to 3′: at least 10, 11, 12, 13, 14, 15, 16, 17, 18, 19, 20 or more contiguous nucleotides complementary to a nucleotide sequence immediately 3′ of the restriction endonuclease site in the recombinant antibody vector; and a 3′ portion of the nucleotide sequence encoding a variable region.

The primers may also comprise one or more nucleotides of the restriction site sequence to ensure that in the expressed antibody, the encoded amino acids are positioned correctly relative to the variable and conserved regions.

In a particular embodiment relating to a recombinant antibody vector comprising a SacI restriction endonuclease site encoding the amino acids EL, PCR primers comprise:
Forward 5′ to 3′: 15 contiguous nucleotides of a nucleotide sequence immediately 5′ of the Sac I restriction endonuclease site in the recombinant antibody vector and the AG dinucleotide of the SacI site; and a 5′ portion of the nucleotide sequence encoding a variable region.
Reverse 5′ to 3′: 15 contiguous nucleotides complementary to a nucleotide sequence immediately 3′ of the SacI restriction endonuclease site in the recombinant antibody vector and the AG dinucleotide of the SacI site; and a 3′ portion of the nucleotide sequence encoding a variable region.

In a preferred embodiment, the nucleotide sequence encoding the immunoglobulin variable region amino acid sequence is inserted into the recombinant antibody vector by way of a ligase independent cloning (LIC) system such as the Clontech In-Fusion™ PCR cloning system. This system obviates the need to include restriction endonuclease sites in the primers used for PCR amplification (i.e. for incorporating 5′ and 3′ restriction endonuclease sites into the PCR amplification product) and the need to partially digest the PCR amplification product with the appropriate restriction endonuclease. Accordingly, the kit of the invention may further comprise an enzyme such as In Fusion™. Other ligase independent cloning systems are known in the art and include, for example, T4 DNA polymerase mediated cloning and ligation-independent cloning of PCR products (LIC-PCR) such as described in Aslanidis & de Jong, 1990 and Aslanidis et al. 1994.

Particular examples of recombinant antibody sequences produced by InFusion™ ligation are provided in the Examples.

In an alternative embodiment, the nucleotide sequence encoding an immunoglobulin variable region amino acid sequence may be ligated into the recombinant antibody vector using a conventional DNA ligase. For example, PCR primers used to amplify a variable region may include restriction endonuclease sites for incorporating 5′ and 3′ restriction endonuclease sites in the PCR amplification product which is then subsequently partially digested with an appropriate restriction endonuclease before ligation into the recombinant antibody vector. According to this embodiment, the kit of the invention may further comprise a DNA ligase.

Following insertion of the amplification product into the recombinant expression vector, a recombinant antibody may be produced that includes no additional amino acid residues other than those provided in the recombinant antibody vector and the variable region.

Suitable host cells for recombinant antibody production may be of eukaryotic or prokaryotic origin, inclusive of bacteria, yeast, plants, insects and animals such as mammals.

For example, gram negative bacteria such as E. coli and gram positive bacteria such as Bacillus species, including but not limited to B. brevis, B. subtilis & B. megaterium, may be used.

Non-limiting examples of yeast cells suitable for recombinant antibody production include Pichia pastoris, Saccaromyces cerevisiae and Ogataea minuta, although without limitation thereto.

Recombinant antibodies may also be produced in transgenic plants or in transgenic plant cell suspension cultures. Non-limiting examples of transgenic plants include species such as Nicotania tabacum, Oryza sativa, Glycine max and Solanum tuberosum. By way of example, plant production of antibodies is reviewed in Hellwig, 2004.

In a preferred embodiment, the host cell is a mammalian cell. Mammalian host cells may include Chinese Hamster Ovary (CHO), HEK293T, NS0, BHK and PER-6 cells, although without limitation thereto. By way of example, mammalian cell production of antibodies is reviewed in Wurm, 2004.

Recombinant antibody expression constructs may be introduced into host cells by “gene transfer” methods that are well known in the art. These include electroporation, DEAE-dextran transfection, calcium phosphate precipitation, cationic liposome-mediated transfection, heat shock and microparticle bombardment, although without limitation thereto. These gene transfer methods may be used to effect stable or transient expression of recombinant antibodies by host cells, as required.

Recombinant antibodies may be isolated, purified or enriched from host cells by any of a variety of techniques well known in the art. These include protein A or protein G purification, ammonium sulphate precipitation and size exclusion chromatography which may be used alone or in combination. Alternatively, recombinant antibodies may comprise a fusion partner amino acid sequence (typically at the C-terminus) to assist purification. Fusion partners include epitope tags (e.g. FLAG, HA, c-myc), or amino acid sequences that assist affinity purification such as metal binding (e.g. 6×His), glutathione binding (e.g. GST) or amylose binding (e.g. MBP) fusion partner sequences.

So that the invention may be readily understood and put into practical effect, reference is made to the following non-limiting examples.

### EXAMPLES

### Example 1

### Design & Construction of Recombinant Antibody Vector

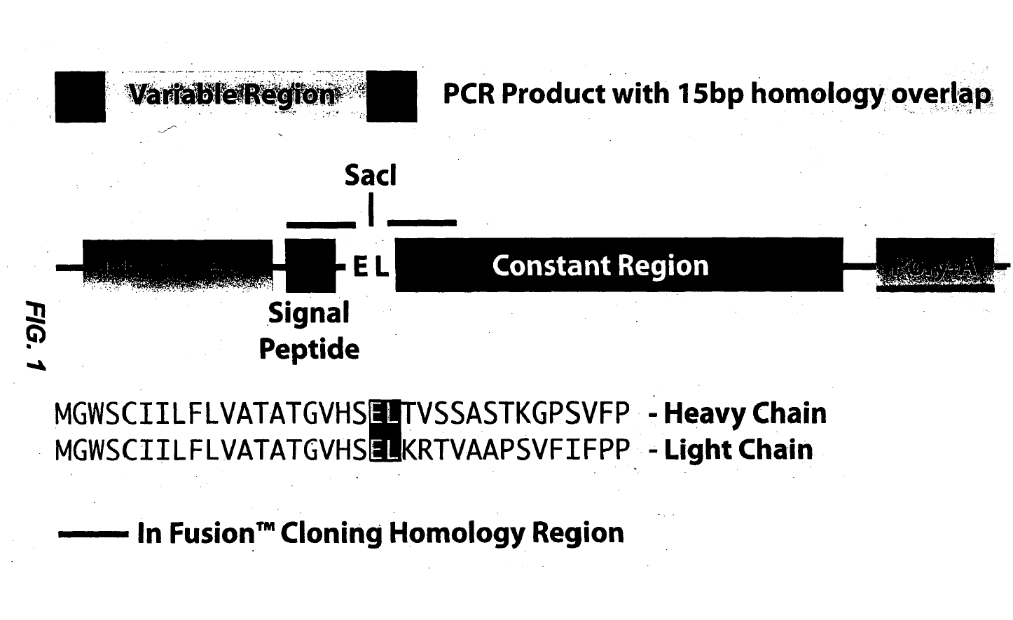

Ig variable regions have been identified that include a glutamate (E) residue at or near the N terminus together with a leucine (L) residue at or near the C-terminus. This feature appears to be present in about 10% of variable regions. By removing the intervening sequence, vector can be constructed which encodes the amino acids EL with the nucleotide sequence GAG CTC thereby forming a SacI site. Insertion of a nucleotide sequence of a variable region into the SacI site results in “splitting” of the EL sequence so that the E residue is at the N terminus of the variable region and the L residue is at the C terminus of the variable region. This avoids addition of extraneous amino acids, the E and L residues being commonly found at or near the N and C termini of variable regions. A schematic description of the “mAbXpress” recombinant antibody vector is provided in FIG. 1.

### Method:

Include a single restriction site after the signal peptide to allow for insertion of variable region. Primers are then designed that are compatible with the In Fusion™ system.
Vector 1: IgG1 HC backbone
Vector 2: IgG4 HC backbone
Vector 3: kappa LC backbone
   Vector 1 and 2: Heavy Chain Sequences without Inserted Variable Region:

Sequence for IgG1 Heavy Chain:

(SEQ ID NO: 2)
MGWSCIILFLVATATGVHSELTVSSASTKGPSVFPLAPSSKSTSGGTAA
LGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPS
VFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNH
YTQKSLSLSPGK*

Sequence for IgG4 Heavy Chain:

(SEQ ID NO: 3)
MGWSCIILFLVATATGVHSELTVSSASTKGPSVFPLAPCSRSTSESTAA
LGCLVKDYFPEPVTVSWNSGALTSGVHTFPAVLQSSGLYSLSSVVTVPS
SSLGTKTYTCNVDHKPSNTKVDKRVESKYGPPCPSCPAPEFLGGPSVFL
FPPKPKDTLMISRTPEVTCVVVDVSQEDPEVQFNWYVDGVEVHNAKTKP
REEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLGK

### Key:

### Signal Peptide

Variable Region with bolded E and L residues

### Constant Region

For Insertion take advantage of single Sac1 site that exists between the 1^{st} residue of the variable region: E and the 5^{th} last residue L

### VHS E-PCR PRODUCT INSERT AND IN FUSION CLONING SITE-L TVSS ASTKG

The EL codons are: GAG CTC and form a SacI site. Primers will then be designed to allow an in frame insertion of the immunoglobulin variable region. This is the same case for both the IgG1 and 4 sequences.

### Vector 3: Light Chain

(SEQ ID NO: 4)
MGWSCIILFLVATATGVHSELKRTVAAPSVFIFPPSDEQLKSGTASVVC
LLNNFYPREAKVQWKVDNALQSGNSQESVTEQDSKDSTYSLSSTLTLSK
ADYEKHKVYACEVTHQGLSSPVTKSFNRGEC*

### Key:

### Signal Peptide

Variable Region with bolded E and L residues

### Constant Region

In Fusion primer vector overlap nucleotides are indicated by italics SacI restriction site nucleotides are bolded
For insertion take advantage of single SacI site that exists between the 1^{st} residue of the variable region: E and the 2^{nd} last residue L

### VHS E-PCR PRODUCT INSERT AND IN FUSION CLONING SITE-L K RTVAA

The EL codons are: GAG CTC and form a SacI site. Primers will then be designed to allow an in frame insertion of the immunoglobulin variable region.

### Primers for the “In Fusion System”:

### Heavy Chain Cloning:

Variable region forward primer to start at amino acid 2 (E) of the variable region.
Reverse primer to be designed so that first bases code for the 5^{th} (L) last amino acid of the variable region
Primers will work with both IgG1 and 4

### IgG HC Cloning Vector:

Vector Sequence with SacI linearization site underlined:

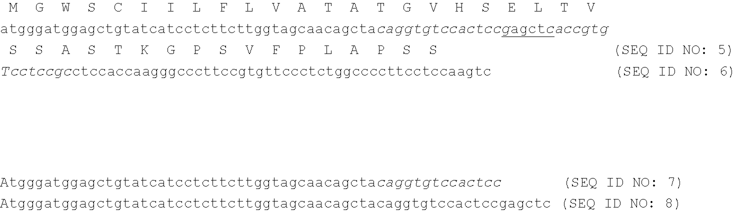

### Required Homology Regions for In Fusion Cloning of IgG1 and 4 HC:

(SEQ ID NO: 9)
For: CAGGTGTCCACTCCG AG Gene Specific Primer
(SEQ ID NO: 10)
Rev: GCGGAGGACACGGTG AG Gene Specific Primer

IgG1 and 4 HC In fusion cloning of variable region example:

Example Gene specific primers for Agen Ab2 HC:

50

(SEQ ID NO: 11)
For: AGGTGCAGCTGAAGGAGTCCGGC
(SEQ ID NO: 12)
Rev: AGTGTGGTGCCCTGGCCCCAGTAG
(Rev complement of: CTACTGGGGCCAGGGCACCACA;
SEQ ID NO: 13)

The AG sequence ensures the E is replaced at the 5′ end and L at the 3′ end

infusion_l:
(SEQ ID NO: 14)
CAGGTGTCCACTCCGAGGTGCAGCTGAAGGAGTCCGGC
infusion_2:
(SEQ ID NO: 15)
GCGGAGGACACGGTGAGTGTGGTGCCCTGGCCCCAGTAG

### Cloning Diagram

### 1. PCR Product.

(SEQ ID NO: 16 )
pcr (+) CAGGTGTCCACTCCGAGGTGC . . . ACCACACTCACCGTGTCCTCCGC
(SEQ ID NO: 17)
pcr (−) GTCCACAGCTGAGGCTCCACG . . . TGGTGTGAGTGGCACAGGAGGCG

### 2. Linearized Vector

(SEQ ID NO: 18)
T G V H S E L T V S S A S
(SEQ ID NO: 19)
vec (+) . . . ACCGCCACCGGAGTGCATTCCGAGCT . . . CACCGTGTCCTCCGCCTCCACCAAGG . . .
(SEQ ID NO: 20)
vec (-) . . . TGGCGGTGGCCTCACGTAAGGC . . . TCGAGTGGCACAGGAGGCGGAGGTGGTTCC . . .

3. Annealing

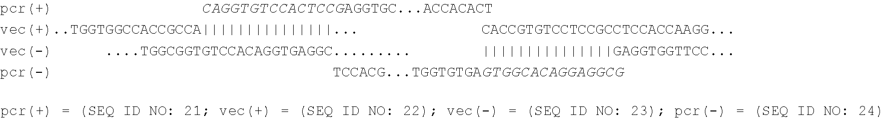

### IgG Kappa LC Cloning Vector:

Vector Sequence with SacI linearization site underlined (SEQ ID NOS:25 & 26):

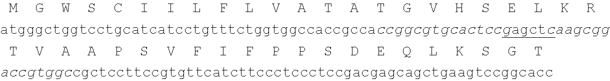

### Required Homology Regions for In Fusion Cloning of Kappa LC:

(SEQ ID NO: 27)
For: CCGGCGTGCACTCCG AG Gene Specific Primer
(SEQ ID NO: 28)
Rev: GCCACGGTCCGCTTG AG Gene Specific Primer

The “AG” dinucleotide after the homology region ensures the insert is placed in frame and maintains the E and L amino acids in the Variable region sequence.

### IgG Kappa LC In Fusion Cloning of Variable Region Example:

Example Gene specific primers for Agen Ab2 Kappa:

(SEQ ID NO: 29)
For: AG ATCGTGATGACCCAGTCCCAG
(SEQ ID NO: 30)
Rev: AG CTCCAGCTTGGTGCCAGCGC
(Rev complement of: GCGCTGGCACCAAGCTGGAG;
SEQ ID NO: 31)

The AG sequence ensures the E is replaced at the 5′ end and L at the 3′ end

### In Fusion Primers: Italics is Vector Overlap and Black is Gene Specific:

infusion_l:
(SEQ ID NO: 32)
CCGGCGTGCACTCCG AGATCGTGATGACCCAGTCCCAG
infusion_2:
(SEQ ID NO: 33)
GCCACGGTCCGCTTG AGCTCCAGCTTGGTGCCAGCGC

### Cloning Diagram

### 1. PCR Product.

per(+)
(SEQ ID NO: 34)
CCGGCGTGCACTCCGAGATCGTGATGACCCAGTCCCAG . . . GCGCTGGCACCAAGCTGGAGCTCAAGCGGACCGTGGC
per(−)
(SEQ ID NO: 35)
GGCCGCACGTGAGGCTCTAGCACTACTGGGTCAGGGTC . . . CGCGACCGTGGTTCGACCTCGAGTTCGCCTGGCACCG

2. Linearized vector

(SEQ ID NO: 36)
T G V H S E L K R T V A A P S
(SEQ ID NO: 37)
vec(+) . . . ACCGCCACCGGAGTGCATTCCGAGCT . . . CAAGCGGACCGTGGCCGCTCCTTCCG . . .
(SEQ ID NO: 38)
vec(-) . . . TGGCGGTGGCCTCACGTAAGGC . . . TCGAGTTCGCCTGGCACCGGCGAGGAAGGC . . .

### 3. Annealing

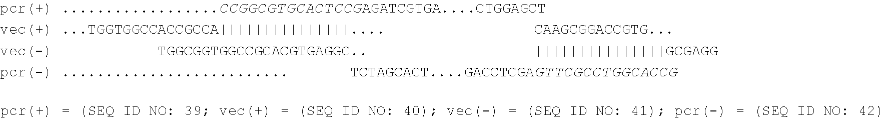

### Example 2

### Production of an Anti-CD83 Recombinant Antibody

### 1. Methods and Materials:

### 1.1. Expression Vector Design.

mAbXpress vectors were assembled using publically available human constant region heavy (IgG1 and IgG4 subtypes) and light chain (K) sequences as described in Example 1. Required DNA was synthesized and codon-optimized for mammalian expression by Geneart AG (Germany). These cassettes were then placed into mammalian expression vectors containing sequences for expression, selection and amplification in mammalian cells (FIG. 1). A single SacI site was included in the expression vector to facilitate linearization and In Fusion™ cloning of the variable region (See section 3 for details).

1.2. Phage Display Panning Against CD83 and Ligation Independent, In Fusion™ Cloning of scFV's.

The extracellular domain of human CD83 was expressed in CHO cells and purified by immobilized metal affinity chromatography. This preparation was used to isolate binders from the Sheets human scFv phage display library (Sheets et al., 1998), kindly provided by Dr James D. Marks (University of California, San Francisco). Several unique binders to recombinant CD83 were isolated, clone 3C12 was selected for cloning and expression.

Variable regions for both the heavy and kappa light chains were PCR amplified from the phagemid vectors using primers against the 5′ and 3′ conserved regions of each chain. An additional 15 by was included on each primer corresponding to upstream and downstream bases of the destination vector to enable ligation independent In Fusion™ cloning. Example primers for the heavy chain were: 3C12_VhFor 5′-CAGGTGTCCACTCCGAGGTGCAGCTGCAGGAG-3′ (SEQ ID NO:43) and 3C12_VhRev 5′-GCGGAGGACACGGTGAGCGTGGTCCCTTGGCCC-3′ (SEQ ID NO:44), and for the kappa chain the primers were: 3C12_VkFor 5′-CCGGCGTGCACTCCGAGATCGTGATGACCCAG-3′ (SEQ ID NO:45) and 3C12_VkRev 5′-GCCACGGTCCGCTTGAGTTCCAGCTTGGTCCC-3′ (SEQ ID NO:46). Underlined regions represent the scFv-specific sequence, which varies from clone to clone. The PCR products were inserted into the mAbXpress heavy and light chain vectors using the In Fusion™ system (Clontech).

### 1.3. Mammalian Cell Expression and Purification

Plasmids were transfected into suspension adapted Chinese Hamster Ovary (CHO) cells using linear PEI-Max (prepared in water) (Polysciences Inc). For transient expression studies, each mL of cells (at 1.5×10⁶ cells/mL) was transfected with 1.6 μg DNA and 5.6 μg PEI, prepared in OptiPro SFM media (Invitrogen). The complex was incubated for 15 mins at room temperature without disruption before addition to the cell suspension. At 4 hours post transfection the cells were diluted by doubling the total volume and IGF-1 was added at 0.1 mg/L before transferring the cultures to 32° C. Secreted antibody was purified using Protein-A chromatography. Purified antibody (3C12) was then analyzed by SDS-PAGE and analytical size exclusion chromatography (SEC) using a BioSep-SEC-53000 (Phenomenex) on an Agilent 1200 series LC. Calibration was done using gel filtration standards (Bio-Rad).

### 1.4. Analysis of Antibody Binding by Flow Cytometery

One million live cells (KH-H2, L428 and FDCP1) were stained with 2.5 μg/mL purified 3C12 mAb or isotype control (human IgG1 κ; Sigma) for 1 hour at 4° C. Bound antibody was detected with a FITC-conjugated anti-human Fc antibody (Cappel, ICN Pharmaceuticals Inc) diluted 1:50 with phosphate buffered saline (PBS). Flow cytometric analysis was performed on a FACS Calibur (Becton Dickinson), and analyzed in FCS Express Version 3 (De Novo Software).

### 1.5. Generation of Lymphokine-Activated Killer (LAK) Cells

Ficol-Paque density gradient separation was used to isolate peripheral blood mononuclear cells (PBMC). NK cells were purified using CD56 Microbeads (Miltenyi) on a VarioMACS separator as per manufacturer's specifications. Cells were cultured in RPMI-10 (100 U/mL penicillin, 100 μg/mL streptomycin, 1×GlutaMAX and 10% fetal calf serum (all from Invitrogen) with 6000 IU/mL human IL-2 (Boehringer Mannheim) at 37° C., 5% CO₂ for 48 hours. Cells were harvested by incubation for 30 mins on ice before supernatant removal, followed by 30 min incubation in ice cold PBS containing 2% EDTA; all harvested cells were washed twice before re-suspension in RPMI-10.

### 1.6. ⁵¹⁻Chromium Release Assay

Functional assays were performed with a CD83⁺ human cell line to determine whether lymphokine activated killer (LAK) cells could induce antibody dependent cellular cytotoxic (ADCC) lysis in the presence of human anti-hCD83 IgG1. KM-H2 cells (1e6 cells/mL) were labeled for 45 mins at 37° C. with 100 μCi ⁵¹Cr in TD buffer (140 mM NaCl, 5 μM KCl, 25 μM Tris-HCl [pH7.4], 0.6 μM Na₂HPO₄, 1% human serum albumin). Cells were washed twice with complete RPMI-10.

5e4 cells/mL LAK cells were plated per well in a V-bottom 96-well plate (Nunc) with 1×10^{3 51}Cr labeled KM-H2 cells. Cells were treated with 5 μg/mL 3C12 or Herceptin (Roche) as a human IgG1 isotype control. Each well contained either 15 μg/mL anti-human CD16 clone 3G8 or mouse IgG1 κ isotype control (both from BD Biosciences) to a final volume of 150 μL. Additional wells containing 1e3 cells/mL KM-H2 cells were prepared with 50 μL RPMI-10 (spontaneous release) or 50 μL 5% Triton-X-100 (total release). Each condition was run with five replicates. Each plate was incubated for 4 hours at 37° C. in 5% CO₂ before centrifugation at 300×g for 5 mins at 24° C. 50 μL supernatant was mixed with 150 μL OptiPhase “SuperMix” and assayed for ⁵¹Cr counts per minute (cpm) with a 1450-MicroBeta scintillation counter (both from Wallac). Specific cell lysis was calculated using the standard formula: % lysis=[(test sample cpm−spontaneous cpm)/(total cpm−spontaneous cpm)*100]. GraphPad Prism Version 5.01 software was used to perform a two way ANOVA.

### 2. Results and Discussion:

The vectors described here (FIG. 1) overcome several major challenges confronting the reformatting of antibody fragments with regards to the insertion of variable sequences into a constant region backbone. Firstly, this method is sequence independent. Since scFv constructs contain semi-conserved framework adjacent to hypervariable regions, the semi-conserved framework sequence can be used as template for PCR. This potentially allows the use of a single primer set to construct the complete, fully assembled antibody. Crucially, this feature means the system is directly applicable to high-throughput applications and automation. Secondly, unlike many reformatting vectors, the site of insertion does not require any extraneous bases. Introduction of such additional amino acids to the primary sequence has the potential to interfere with antibody folding, and/or molecule function, immunogenicity and stability. We have identified semi-conserved glutamate (E) and leucine (L) residues, which are present in many IgGs at the N- and C-termini of the variable region, respectively (FIG. 1). The sequence encoding these two amino acids (GAG CTC) forms the recognition site for the enzyme SacI. This creates an ideal way to linearise the expression vector and facilitate insertion of the variable region PCR product using Ligation Independent Cloning, via the In Fusion™ system (Clontech). This highly efficient method allows for the rapid reformatting of antibodies into the final expression construct.

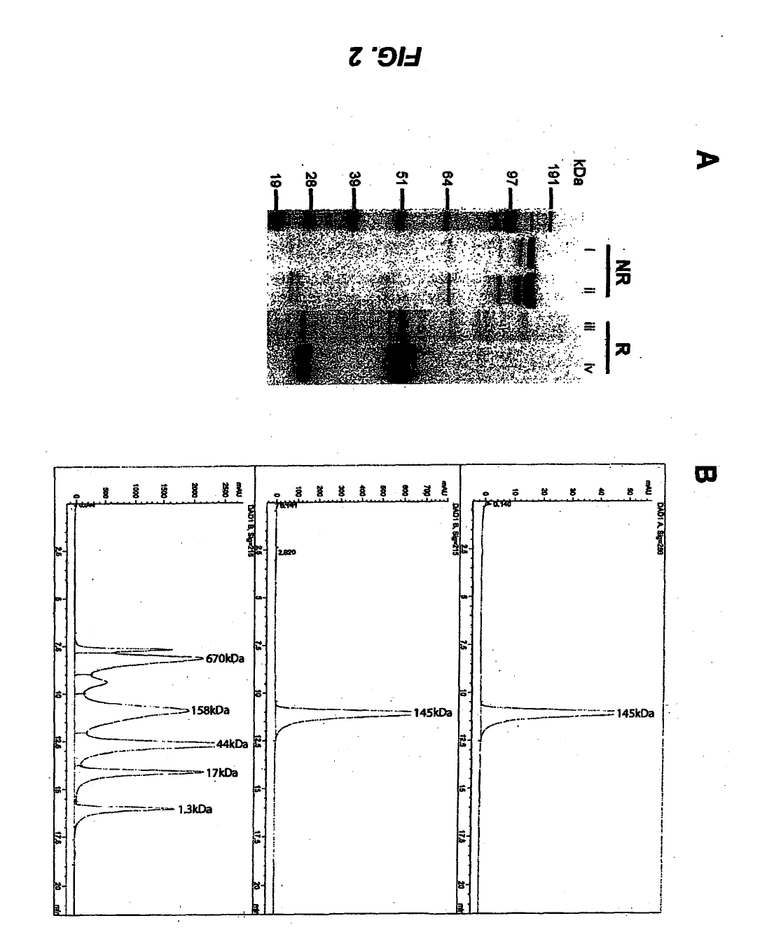

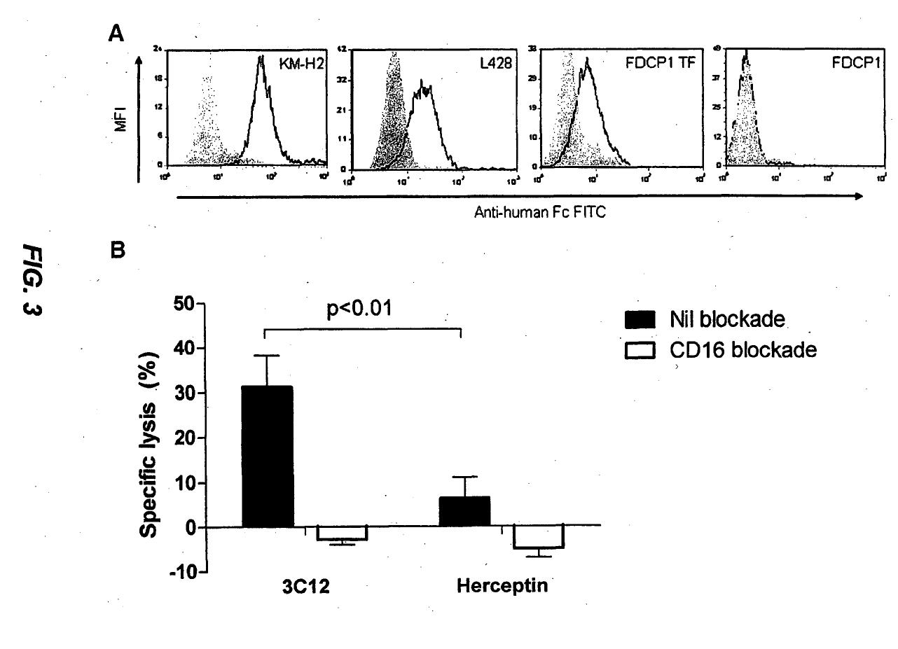

A scFv phage clone was obtained by biopanning a human scFv immunoglobulin gene library (Sheets et al., 1998) three times against recombinant hCD83 extracellular domain (AA1-144). This clone demonstrated specific binding to cell surface CD83 expressed by the human Hodgkin's disease derived cell line, KM-H2 (FIG. 3). Using primers that bind the semi-conserved flanking framework region for each variable region, and which also contain the required vector overlap, this clone was amplified by PCR and cloned into the mAbXpress vectors using the In Fusion™ system (Section 2.2). We expressed of the reformatted IgG1 mAb in CHO cells, followed by protein-A based purification. Analysis by SDS-PAGE and SEC (FIG. 2) showed that molecule was expressed well in our transient expression system, with no observable degradation or aggregation.

In order to show the resulting antibody was functional, we used a purified sample of the recombinant anti-CD83 molecule (3C12 mAb) to demonstrate binding to CD83⁺ human cell lines and hCD83-transfected cells (FIG. 3A). Additionally, in a chromium release functional assay, 3C12 mAb induced significant cytolysis of KM-H2 cells in the presence of activated natural killer (NK) effector cells (FIG. 3B). This antibody-induced lysis, however, is abrogated upon blockade of FcγIIIRa (CD16) with anti-CD16 mAb, 3G8. This indicates the purified 3C12 mAb is capable of mediating ADCC, as the role of FcγIIIRa expression in this mechanism is well characterized (Perussia and Trinchieri, 1984). Additionally, to test the robustness of this system we were also able to repeat this process using a scFv clone isolated from a murine display library, which resulted in the creation of an intact chimeric monoclonal (data not shown).

At present there are no simple, generic methods for reformatting antibody fragments (Fabs, scFvs, dAbs) as complete, fully assembled antibodies. Traditional approaches of antibody reformatting are antibody/laboratory specific and rely on careful, time intensive, sequence analysis and restriction enzyme cutting and ligation mediated cloning. These approaches can also lead to the introduction of extraneous amino acids, which may have profound effects on protein folding and/or bioactivity. Moreover there is limited public availability of the required vectors ((Persic et al., 1997). Here we have described a vector system for the rapid reformatting and expression of functional recombinant monoclonal antibodies that operates essentially independently of the variable region sequence. This is particularly attractive for applications that require cloning of a large number of variable regions during drug discovery and screening.

Throughout this specification, the aim has been to describe the preferred embodiments of the invention without limiting the invention to any one embodiment or specific collection of features. Various changes and modifications may be made to the embodiments described and illustrated herein without departing from the broad spirit and scope of the invention.

All computer programs, algorithms, patent and scientific literature referred to in this specification are incorporated herein by reference in their entirety.

### REFERENCES

Aggarwal, S. (2008) What's fueling the biotech engine-2007. Nature Biotechnology 26, 1227-1233.
Aslanidis C, de Jong P J, Schmitz G. (1994) Minimal length requirement of the single-stranded tails for ligation-independent cloning (LIC) of PCR products PCR Methods Appl. 4172-7.
Aslanidis C & de Jong P J (1990) Ligation-independent cloning of PCR products (LIC-PCR). Nucl. Acids Res. 18 6069-74.
Berger, M., Shankar, V. and Vafai, A. (2002) Therapeutic applications of monoclonal antibodies. Am J Med Sci 324, 14-30.
Coloma, M. J., Hastings, A., Wims, L. A. and Morrison, S. L. (1992) Novel vectors for the expression of antibody molecules using variable regions generated by polymerase chain reaction. J Immunol Methods 152, 89-104.
Hoogenboom, H. R. (2005) Selecting and screening recombinant antibody libraries. Nat Biotechnol 23, 1105-16.
Jostock, T., Vanhove, M., Brepoels, E., Van Gool, R., Daukandt, M., Wehnert, A., Van Hegelsom, R., Dransfield, D., Sexton, D., Devlin, M., Ley, A.,
Hoogenboom, H. and Mullberg, J. (2004) Rapid generation of functional human IgG antibodies derived from Fab-on-phage display libraries. J Immunol Methods 289, 65-80.
Mahler, S. M., Marquis, C. P., Brown, G., Roberts, A. and Hoogenboom, H. R. (1997) Cloning and expression of human V-genes derived from phage display libraries as fully assembled human anti-TNF alpha monoclonal antibodies. Immunotechnology 3, 31-43.
Persic, L., Roberts, A., Wilton, J., Cattaneo, A., Bradbury, A. and Hoogenboom, H. R. (1997) An integrated vector system for the eukaryotic expression of antibodies or their fragments after selection from phage display libraries. Gene 187, 9-18.
Perussia, B. and Trinchieri, G. (1984) Antibody 3G8, specific for the human neutrophil Fc receptor, reacts with natural killer cells. J Immunol 132, 1410-5.
Sheets, M. D., Amersdorfer, P., Finnern, R., Sargent, P., Lindquist, E., Schier, R., Hemingsen, G., Wong, C., Gerhart, J. C. and Marks, J. D. (1998) Efficient construction of a large nonimmune phage antibody library: the production of high-affinity human single-chain antibodies to protein antigens. Proc Natl Acad Sci USA 95, 6157-62.
Wurm, F. M. (2004) Production of recombinant protein therapeutics in cultivated mammalian cells. Nat Biotechnol 22, 1393-8.

1. A recombinant antibody vector comprising: (a) a nucleotide sequence: (i) that comprises a restriction endonuclease site that encodes an amino acid sequence of an immunoglobulin variable region; and (ii) that encodes an immunoglobulin constant region amino acid sequence in the same reading frame as (i), wherein another nucleotide sequence encoding (iii) an immunoglobulin variable region amino acid sequence, is insertable into the restriction endonuclease site in the same reading frame as (ii); and (b) one or more regulatory nucleotide sequences operably linked or connected to said nucleotide sequence.

2. The recombinant antibody vector of claim 1, wherein the amino acid sequence in (i) comprises a plurality of amino acids at least partly conserved in different immunoglobulin variable regions.

3. The recombinant antibody vector of claim 1 or claim 2, wherein the amino acid sequence in (i) comprises a glutamate (E) and/or a leucine (L) residue.

4. The recombinant antibody vector of any preceding claim, wherein the amino acid sequence in (i) is EL.

5. The recombinant antibody vector of any preceding claim, wherein the restriction endonuclease site is a SacI site.

6. The recombinant antibody vector of any preceding claim, wherein the immunoglobulin constant region amino acid sequence in (ii) comprises an immunoglobulin heavy chain constant region amino acid sequence or an immunoglobulin light chain constant region amino acid sequence.

7. The recombinant antibody vector of claim 6, wherein the immunoglobulin constant region amino acid sequence in (ii) is an IgG constant region amino acid sequence.

8. The recombinant antibody vector of any preceding claim, wherein said nucleotide sequence of the recombinant antibody vector further encodes (iv) a signal peptide amino acid sequence.

9. The recombinant antibody vector of any preceding claim, wherein said another nucleotide sequence encoding the immunoglobulin variable region amino acid sequence of (iii) is insertable into the restriction endonuclease site in the recombinant antibody vector in the same reading frame as (ii), without encoding any additional amino acid(s) other than those present in (i), (ii) and (iii).

10. The recombinant antibody vector of claim 9, wherein said another nucleotide sequence in (iii) is insertable into the recombinant antibody vector so that said nucleotide sequence in (a) encodes a contiguous amino acid sequence comprising: a first amino acid of the amino acid sequence in (i), the amino acid sequence of (iii), a second amino acid of the amino acid sequence in (i), and the amino acid sequence of (ii).

11. A kit comprising the recombinant antibody vector of any one of claim 1-10 and one or more reagents for insertion of said another nucleotide sequence encoding an immunoglobulin variable region amino acid sequence into the vector.

12. The kit of claim 11, wherein the one or more reagents include a restriction endonuclease and/or one or more enzymes for insertion of the nucleotide sequence into the vector.

13. The kit of claim 11 comprising an enzyme for ligase independent cloning (LIC) of said another nucleotide sequence of (iii) that encodes the immunoglobulin variable region amino acid sequence into said vector.

14. A method of producing a recombinant antibody expression construct including the step of inserting said another nucleotide sequence of (iii) that encodes an immunoglobulin variable region amino acid sequence into the recombinant antibody expression vector of any one of claims 1-10.

15. The method of claim 14, wherein said another nucleotide sequence of (iii) that encodes the immunoglobulin variable region amino acid sequence is inserted into the recombinant antibody expression vector by ligase independent cloning (LIC).

16. The method of claim 14 or claim 15, wherein the inserted nucleotide sequence of (iii) encodes an immunoglobulin variable region amino acid sequence of an immunoglobulin heavy chain variable region amino acid sequence or of an immunoglobulin light chain variable region amino acid sequence.

17. The method of any one of claims 14 to 16, wherein said another nucleotide sequence of (iii) encodes an amino acid sequence of at least one complementarity determining region (CDR) of the an immunoglobulin variable region.

18. The method of claim 17, wherein said another nucleotide sequence of (iii) encodes respective amino acid sequences of CDR1, CDR2 and CDR3 regions of an immunoglobulin variable region.

19. The method of any one of claims 14 to 18, wherein the immunoglobulin variable region amino acid sequence encoded by said another nucleotide sequence of (iii) is an IgG variable region amino acid sequence.

20. The method of any one of claims 14 to 19, wherein said another nucleotide sequence is inserted into the recombinant antibody vector so that said nucleotide sequence in (a) encodes a contiguous amino acid sequence comprising: a first amino acid of the amino acid sequence in (i), the amino acid sequence of (iii), a second amino acid of the amino acid sequence in (i), and the amino acid sequence of (ii).

21. A recombinant antibody expression construct produced according to the method of any one of claims 14 to 20.

22. A recombinant antibody expression construct comprising the recombinant antibody vector of any one of claims 1-10 and said nucleotide sequence of (iii) encoding the immunoglobulin variable region amino acid sequence, or fragment thereof.

23. The recombinant antibody expression construct of claim 22, wherein the nucleotide sequence of (iii) encodes an immunoglobulin variable region amino acid sequence of an immunoglobulin heavy chain variable region amino acid sequence or of an immunoglobulin light chain variable region amino acid sequence.

24. The recombinant antibody expression construct of claim 23, wherein said another nucleotide sequence of (iii) encodes an amino acid sequence of at least one complementarity determining region (CDR) of the an immunoglobulin variable region.

25. The recombinant antibody expression construct of claim 24, wherein said another nucleotide sequence of (iii) encodes respective amino acid sequences of CDR1, CDR2 and CDR3 regions of an immunoglobulin variable region.

26. The recombinant antibody expression construct of any one of claims 22 to 25, wherein the immunoglobulin variable region amino acid sequence encoded by said another nucleotide sequence of (iii) is an IgG variable region amino acid sequence.

27. The recombinant antibody expression construct of any one of claims 22-26, wherein said nucleotide sequence in (a) encodes a contiguous amino acid sequence comprising: a first amino acid of the amino acid sequence in (i), the amino acid sequence of (iii), a second amino acid of the amino acid sequence in (i), and the amino acid sequence of (ii).

28. A host cell comprising the recombinant antibody vector of any one of claims 1-10 or the recombinant antibody expression construct of any one of claims 21 to 27.

29. The host cell of claim 28 which is plant cell, a bacterial cell, a yeast cell or a mammalian cell.

30. A method of producing a recombinant antibody including the step of isolating, purifying or enriching a recombinant antibody from the host cell of claim 28 or claim 29.

31. A recombinant antibody encoded by the recombinant antibody expression construct of any one of claims 21 to 28 or produced according to the method of claim 30.


SEQUENCE LISTING (46 sequences)

SEQ ID NO: 1 - DNA, 6 bp, Artificial sequence
  Sac1 cleavage site
gagctc

SEQ ID NO: 2 - protein, 355 aa, Homo sapiens
MGWSCIILFLVATATGVHSELTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 352 aa, Homo sapiens
MGWSCIILFLVATATGVHSELTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 129 aa, Homo sapiens
MGWSCIILFLVATATGVHSELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 40 aa, Artificial sequence
  Vector sequence
MGWSCIILFLVATATGVHSELTVSSASTKGPSVFPLAPSS

SEQ ID NO: 6 - DNA, 125 bp, Artificial sequence
  Vector nucleotide sequence
atgggatggagctgtatcatcctcttcttggtagcaacagctacaggtgtccactccgagctcaccgtgtcctccgcctccaccaagggcccttccgtgttccctctggccccttcctccaagtc

SEQ ID NO: 7 - DNA, 57 bp, Artificial sequence
  Vector nucleotide sequence
atgggatggagctgtatcatcctcttcttggtagcaacagctacaggtgtccactcc

SEQ ID NO: 8 - DNA, 63 bp, Artificial sequence
  vector nucleotide sequence
atgggatggagctgtatcatcctcttcttggtagcaacagctacaggtgtccactccgagctc

SEQ ID NO: 9 - DNA, 15 bp, Artificial sequence
  Primer
caggtgtccactccg

SEQ ID NO: 10 - DNA, 15 bp, Artificial sequence
  Primer
gcggaggacacggtg

SEQ ID NO: 11 - DNA, 23 bp, Artificial sequence
  Primer
aggtgcagctgaaggagtccggc

SEQ ID NO: 12 - DNA, 24 bp, Artificial sequence
  Primer
agtgtggtgccctggccccagtag

SEQ ID NO: 13 - DNA, 22 bp, Artificial sequence
  Pirmer
ctactggggccagggcaccaca

SEQ ID NO: 14 - DNA, 38 bp, Artificial sequence
  Cloning sequence
caggtgtccactccgaggtgcagctgaaggagtccggc

SEQ ID NO: 15 - DNA, 39 bp, Artificial sequence
  Cloning sequence
gcggaggacacggtgagtgtggtgccctggccccagtag

SEQ ID NO: 16 - DNA, 44 bp, Artificial sequence
  PCR product
caggtgtccactccgaggtgcaccacactcaccgtgtcctccgc

SEQ ID NO: 17 - DNA, 44 bp, Artificial sequence
  PCR product
gtccacagctgaggctccacgtggtgtgagtggcacaggaggcg

SEQ ID NO: 18 - protein, 13 aa, Artificial sequence
  Vector peptide
TGVHSELTVSSAS

SEQ ID NO: 19 - DNA, 52 bp, Artificial sequence
  Vector sequence
accgccaccggagtgcattccgagctcaccgtgtcctccgcctccaccaagg

SEQ ID NO: 20 - DNA, 52 bp, Artificial sequence
  Vector sequence
tggcggtggcctcacgtaaggctcgagtggcacaggaggcggaggtggttcc

SEQ ID NO: 21 - DNA, 29 bp, Artificial sequence
  PCR product
caggtgtccactccgaggtgcaccacact

SEQ ID NO: 22 - DNA, 41 bp, Artificial sequence
  Vector sequence
tggtggccaccgccacaccgtgtcctccgcctccaccaagg

SEQ ID NO: 23 - DNA, 33 bp, Artificial sequence
  vector sequence
tggcggtgtccacaggtgaggcgaggtggttcc

SEQ ID NO: 24 - DNA, 29 bp, Artificial sequence
  PCR product
tccacgtggtgtgagtggcacaggaggcg

SEQ ID NO: 25 - DNA, 132 bp, Artificial sequence
  Vector sequence with restriction site
atgggctggtcctgcatcatcctgtttctggtggccaccgccaccggcgtgcactccgagctcaagcggaccgtggccgctccttccgtgttcatcttccctccctccgacgagcagctgaagtccggcacc

SEQ ID NO: 26 - protein, 43 aa, Artificial sequence
  Vector encoded peptide
MGWSCIILFLVATATGVHSELKRVAAPSVFIFPPSDEQLKSGT

SEQ ID NO: 27 - DNA, 15 bp, Artificial sequence
  PCR primer
ccggcgtgcactccg

SEQ ID NO: 28 - DNA, 15 bp, Artificial sequence
  PCR primer
gccacggtccgcttg

SEQ ID NO: 29 - DNA, 23 bp, Artificial sequence
  PCR primer
agatcgtgatgacccagtcccag

SEQ ID NO: 30 - DNA, 22 bp, Artificial sequence
  PCR primer
agctccagcttggtgccagcgc

SEQ ID NO: 31 - DNA, 20 bp, Artificial sequence
  PCR primer
gcgctggcaccaagctggag

SEQ ID NO: 32 - DNA, 38 bp, Artificial sequence
  PCR primer
ccggcgtgcactccgagatcgtgatgacccagtcccag

SEQ ID NO: 33 - DNA, 37 bp, Artificial sequence
  PCR primer
gccacggtccgcttgagctccagcttggtgccagcgc

SEQ ID NO: 34 - DNA, 75 bp, Artificial sequence
  PCR product
ccggcgtgcactccgagatcgtgatgacccagtcccaggcgctggcaccaagctggagctcaagcggaccgtggc

SEQ ID NO: 35 - DNA, 75 bp, Artificial sequence
  PCR product
ggccgcacgtgaggctctagcactactgggtcagggtccgcgaccgtggttcgacctcgagttcgcctggcaccg

SEQ ID NO: 36 - protein, 15 aa, Artificial sequence
  Vector peptide
TGVHSELKRTVAAPS

SEQ ID NO: 37 - DNA, 52 bp, Artificial sequence
  Vector sequence
accgccaccggagtgcattccgagctcaagcggaccgtggccgctccttccg

SEQ ID NO: 38 - DNA, 52 bp, Artificial sequence
  Vector sequence
tggcggtggcctcacgtaaggctcgagttcgcctggcaccggcgaggaaggc

SEQ ID NO: 39 - DNA, 52 bp, Artificial sequence; identical to SEQ ID NO: 38
  PCR product

SEQ ID NO: 40 - DNA, 28 bp, Artificial sequence
  Vector sequence
tggtggccaccgccacaagcggaccgtg

SEQ ID NO: 41 - DNA, 28 bp, Artificial sequence
  Vector sequence
tggcggtggccgcacgtgaggcgcgagg

SEQ ID NO: 42 - DNA, 32 bp, Artificial sequence
  PCR product
tctagcactgacctcgagttcgcctggcaccg

SEQ ID NO: 43 - DNA, 32 bp, Artificial sequence
  PCR primer
caggtgtccactccgaggtgcagctgcaggag

SEQ ID NO: 44 - DNA, 33 bp, Artificial sequence
  PCR primer
gcggaggacacggtgagcgtggtcccttggccc

SEQ ID NO: 45 - DNA, 32 bp, Artificial sequence
  PCR primer
ccggcgtgcactccgagatcgtgatgacccag

SEQ ID NO: 46 - DNA, 32 bp, Artificial sequence
  PCR primer
gccacggtccgcttgagttccagcttggtccc